(19) United States
(12) Patent Application Publication
(10) Pub. No.: US 2008/0110961 A1
(43) Pub. Date: May 15, 2008

(54) Initiator Coating of Staples

(75) Inventors: James W. Voegele, Cincinnati, OH (US); Frederick E. Shelton, Hillsboro, OH (US); Stephen E. Eichmann, Palo Alto, CA (US); Mark S. Ortiz, Milford, OH (US)

Correspondence Address: FROST BROWN TODD, LLC, 2200 PNC CENTER, 201 E. FIFTH STREET, CINCINNATI, OH, 45202, US

(73) Assignee: Ethicon Endo-Surgery, Inc., Cincinnati, OH (US)

(21) Appl. No.: 11/558,497

(22) Filed: Nov. 10, 2006

(51) Int. Cl. A61B 17/115; A61B 17/08

(52) U.S. Cl. 227/179.1; 606/214; 606/220

(57) ABSTRACT

Provided are surgical fasteners coated with a material including an initiator configured for activation upon application of an initiator. The surgical fasteners may be configured for use in combination with a surgical fastener applying apparatus. In one version, the surgical fastener is a surgical staple including a pair of legs, a crown interconnecting the pair of legs, and a material including an initiator coating at least a portion of the legs and/or crown. Adhesive may be delivered into contact with the initiator associated with the surgical fasteners such that a desirable size, shape, and configuration of activated or polymerized adhesive is created.

### FIELD OF THE INVENTION

The present invention relates, in general, to tissue fastening devices, and more particularly, to tissue fastening devices utilizing a combination of staples and adhesives.

### BACKGROUND OF THE INVENTION

Coating medical instruments with various materials for various purposes is a technique well known in the art. In particular, adhesives and sealants have been used to supplement or replace staple based transaction devices for many years. The primary challenges in accomplishing this are controlling and guiding the adhesive into the correct location at the correct time and preventing the adhesive from adhering to the stapler itself or to the treatment site.

Generally, coatings are often applied to create a water absorbent and lubricious coating for surgical instruments and for in-dwelling biomaterials such as stents, screws, internal splints, tubing, catheters, wire guides, and the like. Such coatings often minimize the trauma of contact of the medical device with tissue and biological fluids. In particular, coatings have been used to provide a slippery and lubricious coating for reducing the coefficient of friction of a surface of a medical device to facilitate movement and maneuverability of the device. Lubricious coatings made from hydrophilic polymers are well-known in the art.

Medical devices such as surgical fasteners and staples often replace suturing when joining or anastomosing various body structures such as, for example, the bowel or bronchus. The surgical stapling devices used to apply surgical staples are generally designed to simultaneously cut and seal an extended segment of tissue in a patient. Linear or annular surgical stapling devices are often employed by surgeons to sequentially or simultaneously apply one or more linear rows of surgical fasteners, such as staples or two-part fasteners, to body tissue for the purpose of joining segments of body tissue together and/or for the creation of anastomosis. Linear surgical stapling devices generally include a pair of jaws or finger-like structures between which body tissue to be joined is placed. When the surgical stapling device is actuated and/or “fired,” firing bars generally move longitudinally and contact staple drive members in one of the jaws such that surgical staples are pushed through the body tissue and into/against an anvil in the opposite jaw, thereby crimping the staples closed. A knife blade may be provided to cut between the rows/lines of staples. Examples of such linear surgical stapling devices are Models “GIA™”, “Endo GIA™” and “Premium Multi-fire TA™” instruments available from United States Surgical, a Division of Tyco Health-Care Group, LP, Norwalk, Conn. and disclosed in, inter alia, U.S. Pat. No. 5,465,896 to Allen et al, U.S. Pat. No. 6,330,965 to Milliman et al., and U.S. Pat. No. 6,817,508 to Racenet et al., the contents of each of which are incorporated herein by reference.

Another type of surgical stapler is an end-to-end anastomosis stapler. An example of such a device is a Model “EEA™” instrument available from United States Surgical, a Division of Tyco Health-Care Group, LP, Norwalk, Conn. and disclosed in U.S. Pat. No. 5,392,979 to Green et al., the contents of which is incorporated herein by reference. In general, an end-to-end anastomosis stapler typically places an array of staples into the approximated sections of a patient's bowels or other tubular organs. The resulting anastomosis contains an inverted section of bowel which contains numerous “B” shaped staples to maintain a secure connection between the approximated sections of bowel.

Sealants, such as biological sealants, may also be applied to a surgical site to guard against leakage. Typically, the biological sealants are manually applied to the outer surface of the staple line by a physician by spraying on, brushing on, swabbing on, or any combinations thereof. This manual application of biological sealant may lead to non-uniformity of the thickness of sealant across the staple line and/or omitting a portion of the intended coverage area due to the inability to see or reach the desired location. Additionally, the agglutinative nature of the adhesive may cause it to stick to and clog medical instruments reducing or eliminating their functionality.

It would therefore be advantageous to provide a fastener that exhibits the short term benefits of an adhesive and the long term benefits of a mechanical fastener. It would be further advantageous to provide the short term benefits of the adhesive without creating superfluous amounts of adhesive. It would also be advantageous to provide a surgical device configured to staple and cut tissue that may be used simply and easily in combination with an adhesive initiator at desired sites to attract and set an adhesive about a junction or cut line in the tissue to prevent, for example, unwanted adhesive migration away from the desired adhesion areas.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a perspective view of one version of a surgical fastener shown coated with an adhesive initiator.

FIG. 2 is a partial perspective view of tissue having a plurality of fasteners applied thereto, where the fasteners are shown during application of an adhesive thereto.

FIG. 3 is a partial perspective view of a tissue segment having a plurality of fasteners applied thereto, where the fasteners are shown following application and initiation of the adhesive.

FIG. 4 is a cross sectional view, taken along line 4-4 of FIG. 3, of the tissue segment having a plurality of fasteners applied thereto, showing one version of a relationship between the fasteners, the adhesive, the adhesive initiator, and tissue.

FIG. 5 is a partial perspective view of a tissue segment having a plurality of fasteners applied thereto, showing an alternate version of a relationship between the fasteners, the adhesive, the adhesive initiator, and tissue.

### DETAILED DESCRIPTION OF THE INVENTION

The following description of certain examples of the invention should not be used to limit the scope of the present invention. As will be realized, the invention is capable of other different and obvious aspects, all without departing from the invention. Accordingly, the drawings and descriptions should be regarded as illustrative in nature and not restrictive. Versions will now be described in detail with reference to the drawing figures wherein like reference numerals identify similar or identical elements. As used herein and as is traditional, the term “distal” refers to that portion which is farthest from the user while the term “proximal” refers to that portion which is closest to the user.

In one version, adhesives having advantageous short term bonding and sealing characteristics are combined with surgical staples or fasteners having advantageous long term bonding characteristics. In one version, the adhesives used in combination with the fasteners are initiated only upon contact with an initiator, where the initiator is doped, impregnated, or otherwise associated with the fastener. The adhesives may be configured such that initiation thereof occurs only when the adhesive comes into contact or close proximity to the fastener containing the initiator. In such a manner, excessive adhesive that has not been activated, and therefore does not exhibit agglutinative properties, may be washed from a surgical site. Providing an adhesive that is readily removable when superfluous or excessive may provide the benefits of an adhesive without the drawbacks of imprecise and excessive adhesive delivery and solidification.

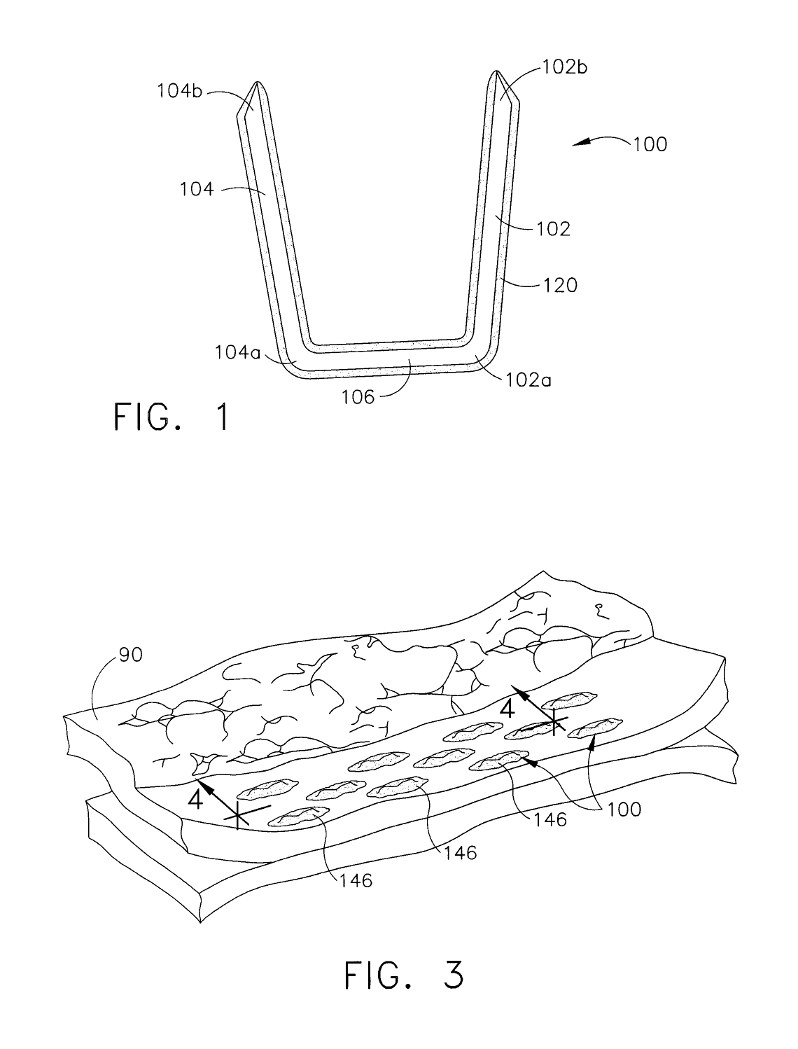

With reference to FIG. 1, a surgical fastener, in the form of a surgical staple, is generally shown as 100. Surgical staples of the present disclosure typically include any metallic staple used to join together tissue parts and/or adjacent tissues. Surgical staples 100 may be made of metal such as, for example, stainless steel or titanium, or any other material known by one having skill in the art. For example, surgical staples 100 may also be fabricated from bio-absorbable material, or the like.

Bio-absorbable materials used for surgical staples 100 include, but are not limited to, those fabricated from homopolymers, copolymers or blends obtained from one or more monomers such as glycolide, glycolic acid, lactide, lactic acid, p-dioxanone, α-prolactone, trimethylene carbonate, and combinations thereof. Other bio-absorbable materials include polyglycolic acid (PGA) and polylactic acid (PLA).

With continued reference to FIG. 1, surgical staple 100 includes a pair of legs 102, 104 which are interconnected to one another by a crown or backspan 106 extending between first ends 102a, 104a, respectively, thereof. As seen in FIG. 1, the crown 106 is substantially perpendicular to legs 102, 104. However, it is envisioned that the crown 106 may take on any shape or configuration as needed or desired and may have any orientation relative to legs 102, 104. For example, the crown 106 may include two sections which extend angularly from legs 102, 104 and are connected at an apex (not shown).

As seen in FIG. 1, respective distal ends 102b, 104b of legs 102, 104 are sharpened to facilitate penetration of legs 102, 104 into tissue, or the like. In the illustrated version, the surgical staple 100 is coated with a material 120. It is envisioned that the material 120 may be applied to the entirety of the surgical staple 100, as shown in FIG. 1, or may be applied to regions of the surgical staple 100 as desirable. For example, the material 120 may be applied solely to legs 102, 104, solely to one of legs 102, 104, solely to the crown 106, and/or to any other portion thereof. It is further contemplated that the material 120 may be doped or impregnated into the legs 102, 104, the crown 106, and/or any other portion of the surgical staple 100.

In one version, the surgical staples 100 may be fabricated from a bio-absorbable material that is impregnated with the material 120. Accordingly, in use, the material 120 of surgical staples 100 may function to retard any bleeding that may occur from the tissue, in the manner of a sealant, and to secure the approximated tissue together, in the manner of an adhesive. The bio-absorbability of one version of the surgical staples 100 may allow for at least a portion of the surgical staples 100 to be absorbed into the body after a predetermined amount of time. For example, surgical staples 100 may remain in place in the body for approximately 2-3 weeks in order for the anastomosis to sufficiently heal prior to absorption.

As discussed herein, it is envisioned that the surgical staples 100 may be impregnated with a material 120 which is a pre-cured adhesive or sealant. The pre-cured sealant or adhesive may react with the moisture and/or heat of the body tissue to thereby activate the sealing and/or adhesive properties of the sealant or adhesive. It is envisioned that the pre-cured sealant or adhesive may be a hydro-gel or the like.

It is contemplated that the material 120 may include any suitable material for joining, healing, sealing, or otherwise treating tissue. In one version, the material 120 is a bio-compatible sealant including, but not limited to, sealants that cure upon tissue contact, sealants that cure upon exposure to ultraviolet (UV) light, sealants that are two-part systems that become activated when combined, or combinations thereof. Any known suitable adhesive may be used. In one version, it is contemplated that such sealants and/or adhesives are curable. For example, sealants having a cure time of from about ten to fifteen seconds may be used. The sealant and/or adhesive may be bioabsorbable and/or a bio-resorbable material. In an alternate version, a sealant and/or adhesive having a cure time of about 30 seconds may be provided. It is further contemplated that the material 120 may be a pre-cured adhesive or sealant.

The material 120 includes a sealant. Such a sealant may be, for example, a PEG-based material. Examples of classes of materials that may be provided as the sealant and/or adhesive include, but are not limited to, acrylate or methacrylate functional hydrogels in the presence of a biocompatible photoinitiator, alkyl-cyanoacrylates, isocyanate functional macromers with or without amine functional macromers, succinimidyl ester functional macromers with amine or sulfhydryl functional macromers, epoxy functional macromers with amine functional macromers, mixtures of proteins or polypeptides in the presence of aldehyde crosslinkers, genipin, or water-soluble carbodiimides, anionic polysaccharides in the presence of polyvalent cations, or combinations thereof. Materials that may be utilized further include isocyanate terminated hydrophilic urethane prepolymers derived from organic polyisocyanates and oxyethylene-based diols or polyols, including those disclosed in U.S. Pat. Nos. 6,702,731 and 6,296,607 and U.S. Published Patent Application No. 2004/0068078; alpha-cyanoacrylate based adhesives including those disclosed in U.S. Pat. No. 6,565,840; alkyl ester based cyanoacrylate adhesives including those disclosed in U.S. Pat. No. 6,620,846; adhesives based on biocompatible cross-linked polymers formed from water soluble precursors having electrophilic and nucleophilic groups capable of reacting and cross-linking in situ, including those disclosed in U.S. Pat. No. 6,566,406; two part adhesive systems including those based upon polyalkylene oxide backbones substituted with one or more isocyanate groups in combination with bioabsorbable diamine compounds, or polyalkylene oxide backbones substituted with one or more amine groups in combination with bioabsorbable diisoyeanate compounds as disclosed in U.S. Published Patent Application No. 2003/0032734, the contents of which are incorporated by reference herein; and isocyanate terminated hydrophilic urethane prepolymers derived from aromatic diisocyanates and polyols as disclosed in U.S. Published Patent Application No. 2004/0115229, the contents of which are incorporated by reference herein.

It is envisioned and within the scope of the present disclosure that the material 120 may include one or a combination of adhesives, hemostats, sealants, or any other tissue or wound-treating material. Surgical biocompatible materials 120 that may be used in accordance with the present disclosure include adhesives whose function is to attach or hold structures, sealants to prevent fluid leakage, and hemostats to halt or prevent bleeding. Examples of adhesives that may be employed include protein derived, aldehyde-based adhesive materials such as, for example, the commercially available albumin/glutaraldehyde materials sold under the trade designation BioGlue™ by Cryolife, Inc., and cyanoacrylate-based materials sold under the trade designations hidermil™ and Derma Bond™ by Tyco Healthcare Group, LP and Ethicon Endo-Surgery, Inc., respectively.

Examples of sealants that may be provided include fibrin sealants, collagen-based sealants, and synthetic polymer-based tissue sealants. Examples of commercially available sealants are synthetic polyethylene glycol-based, hydrogel materials sold under the trade designation CoSeal™ by Cohesion Technologies and Baxter International, Inc. Examples of hemostat materials that may be provided include fibrin-based, collagen-based, oxidized regenerated cellulose-based, and gelatin-based topical hemostats, as well as aluminum alum (i.e., ammonium alum or aluminum ammonium sulfate). Examples of commercially available hemostat materials are fibrinogen-thrombin combination materials sold under the trade designations CoStasis™ by Tyco Healthcare Group, LP, and Tisseel™ sold by Baxter International, Inc. Hemostats herein include astringents, e.g., aluminum sulfates, and coagulants. A further example of a hemostat includes “Quick Clot™”, commercially available from Z-Medica, Inc., Newington, Conn. [00037] The medicament may include one or more medically and/or surgically useful substances such as drugs, enzymes, growth factors, peptides, proteins, dyes, pigments, diagnostic agents or hemostasis agents, monoclonal antibodies, or any other pharmaceutical used in the prevention of stenosis. The medicament may be disposed on structure 100 or impregnated into structure 100.

The material 120 may include visco-elastic film forming materials, cross-linking reactive agents, and energy curable adhesives. It is envisioned that the material 120 and, in particular, adhesive, may be cured with the application of water and/or glycerin (1,2,3,-pranatetriol, also known as glycerol or glycerine) thereto. In this manner, the water and/or glycerin may cure the adhesive and hydrate the wound.

It is further contemplated that the material 120 may include, for example, compositions and/or compounds that accelerate or beneficially modify the healing process when particles of the composition and/or compound are applied to or exposed to a surgical repair site. For example, material 120 may be a therapeutic agent that will be deposited at the repair site. The therapeutic agent may be chosen for its antimicrobial properties, capability for promoting repair or reconstruction and/or new tissue growth. For example, the material 120 may comprise “SilvaSorb™”, commercially available from AcryMed, Lie, Portland, Oreg. Antimicrobial agents such as broad spectrum antibiotic (gentamycin sulfate, erythromycin or derivatized glycopeptides) which are slowly released into the tissue may be applied in this manner to aid in combating clinical and sub-clinical infections in a tissue repair site. To promote repair and/or tissue growth, the material 120 may include one or several growth promoting factors including, for example, fibroblast growth factor, bone growth factor, epidermal growth factor, platelet derived growth factor, macrophage derived growth factor, alveolar derived growth factor, monocyte derived growth factor, magainin, and the like. Therapeutic indications include glycerol with tissue or kidney plasminogen activator to cause thrombosis, superoxide dimutase to scavenge tissue damaging free radicals, tumor necrosis factor for cancer therapy or colony stimulating factor and interferon, interleukin-2, or other lymphokine to enhance the immune system. It is further envisioned and within the scope of the present disclosure for the material 120 to include any microbial agent, analgesic, growth factor, and anti-inflammatory agent known by one having skill in the art or any combination thereof.

Those skilled in the art will recognize that the successful surface treatment of surgical staple 100, prior to the application of the material 120 may include pre-cleaning the surgical staple 100 and controlling the moisture at the surface of the surgical staple 100 in order to ensure complete and/or proper coating of the surgical staple 100. Multi-step cleaning and drying operations may therefore be used to provide a clean surface and/or to control moisture. Once the surface of the surgical staple 100 is treated, a solution containing the material 120 may be applied to the treated surgical staple 100.

Still referring to FIG. 1, it is contemplated and within the scope of the present disclosure for any of the surgical staples 100 disclosed herein to have equal length legs, unequal length legs, a relatively short crown as compared to the length of the legs, a relatively long crown as compared to the length of the legs, a symmetrical transverse cross-sectional profile in at least one of the legs and the crown, and an asymmetrical transverse cross-sectional profile in at least one of the legs and the crown. For example, each leg and/or the crown may have a cross-sectional profile which is polygonal, such as, triangular, rectangular, hexagonal or any combination thereof or the like. Moreover, each leg and/or the crown may have a cross-sectional profile that is circular, ovular, or the like. It is further envisioned that the crown may be either linear or non-linear. In one version, the surgical staples 100 include legs that do not lie in the same plane as one another. In other words, one leg and the crown of the surgical staple 100 define a first plane, and the other leg of the surgical staple 100 lies in a second plane which is non-coplanar, or transverse to the first plane.

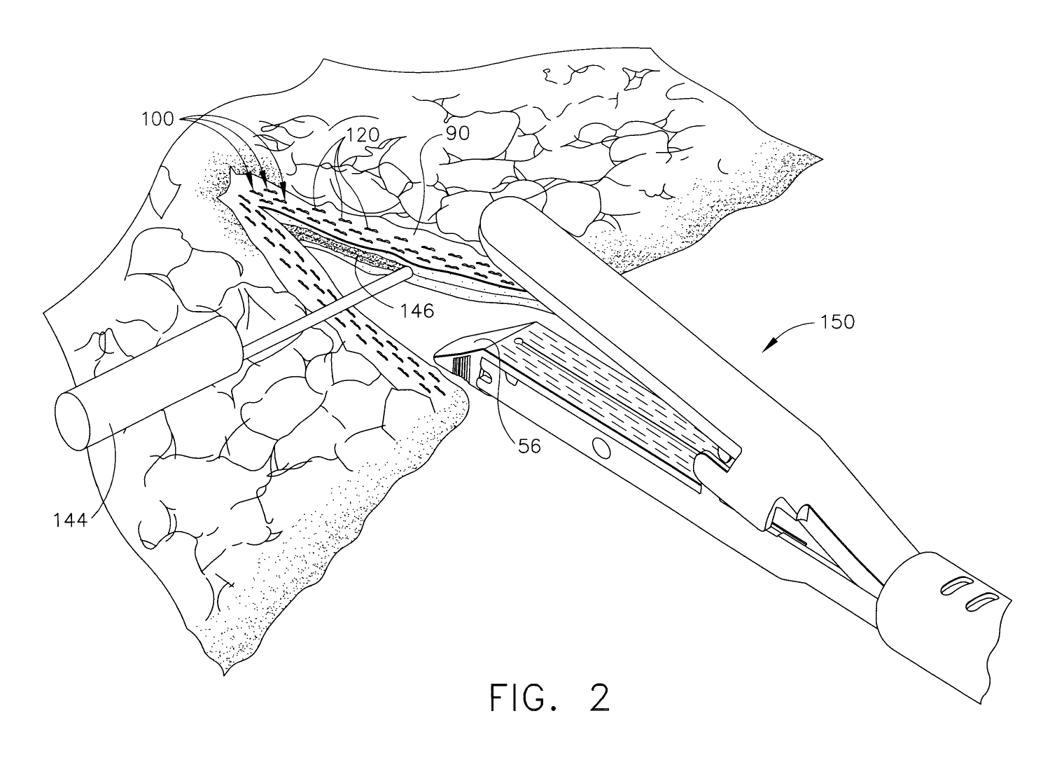

Referring to FIG. 2, a partial perspective view of a tissue segment 90 is shown after the application of a plurality of surgical staples 100. Prior to delivery, the surgical staples 100 may be retained within a removable cartridge 56 in an array of six parallel longitudinal rows. In the illustrated version, the rows of staples 100 are staggered. A knife (not shown) may be located between the innermost rows of the staples 100 while in the removable cartridge 56 and, when the surgical staple delivery instrument 150 is fired, the knife may travel distally along the longitudinal axis thereof to cut the tissue 90. FIG. 2 shows the tissue 90 after being clamped, stapled, and cut with the surgical stapler delivery instrument 150. As illustrated, the firing process has formed six staggered rows of staples 100 in the compressed tissue 90 and has cut the tissue 90. In FIG. 2, an adhesive applicator 144 is shown applying adhesive 146 along the cut tissue 90 to ensure, for example, pneumostasis and hemostasis along the cut tissue. As the adhesive 146 contacts the material 120 having an adhesive initiator, the adhesive 146 may polymerize or set, thereby bonding the cut tissue to create a seal.

The surgical staple delivery instrument 150 may be any suitable fastener delivery instrument or device including linear-type surgical staplers, non-linear-type surgical staplers, annular-type surgical staplers, endoscopic-type surgical staplers, skin-type surgical staplers, or the like. The surgical staple delivery instrument 150 may be, for example, that disclosed in U.S. Pat. No. 5,597,107 to Knodel, which is herein incorporated by reference in its entirety. Surgical stapling devices are well known in the art for clamping onto tissue and placing a plurality of fasteners in an array into the tissue. In one version, a knife (not shown) is included in the surgical stapling device that is configured to sever or cut tissue within the array of staples. Such stapling devices may be circular, linear, arcuate, or any other shape and may be used, for example, to resect or transect tissue, to perform an anastomosis on luminal structures such as intestines, to resect lung tissue, or for any other suitable purpose.

Still referring to FIG. 2, shown is an adhesive applicator 144, which may be any suitable delivery instrument or device including, for example, a syringe. The adhesive applicator 144 may be filled with an adhesive 146 and may be operably configured to deliver the adhesive 146 as desired by the clinician. The adhesive 146 includes, for example, polymerizable and/or cross-linkable materials such as a cyanoacrylate adhesive. The adhesive 146, for example, may be a monomeric (including prepolymeric) adhesive composition, a polymeric adhesive composition, or any other compound that can adhere to tissue. In embodiments, the monomer may be a 1,1-disubstituted ethylene monomer such as, for example, an alpha-cyanoacrylate. When cross linked or polymerized, the cyanoacrylate can change from a liquid to a solid. Polymerized adhesives, for example, may be formulated to be flexible to rigid. If desired, the adhesive 146 may be a single part or dual part adhesive, and/or may contain additives such as alternate compounds. Polymerization of the adhesive 146 may occur from association with the material 120 associated with the surgical staples 100 containing an initiator.

The surgical staples associated with the material 120, containing an initiator, may be adhesively inert prior to application of the adhesive 146. In this manner, surgical staples 100 having advantageous adhesive qualities may be applied to tissue using standard surgical staple delivery instruments 150. Once the surgical staples 100 are applied to tissue, a polymerized adhesive may be created by providing an uninitiated adhesive to regions containing the material 120. Administering an adhesive to pre-placed surgical staples 100 may provide the benefits of an adhesive, including reduced blood loss and improved healing, without requiring specialized surgical staple delivery instruments or highly sophisticated fasteners. Additionally, providing an adhesive after pre-placement of the surgical staples 100 may polymerize the adhesive at exposed locations only, thereby minimizing the unnecessary polymerization of adhesive, for example, within, on, or around tissue or instruments.

Still referring to FIG. 2, the adhesive 146 is shown being administered to the tissue 90 between two tissue sections. In this manner, the adhesive may be delivered into proximity with the surgical staples 100 to form a polymerized adhesive. The adhesive may be delivered adjacent the crown 106, the legs 102, 104, or any other suitable portion of a surgical staple or fastener comprising the material 120 having an initiator associated therewith. The surgical staple 100 containing the adhesive initiator may be configured to initiate setting or polymerization of an adhesive 146, where the adhesive 146 includes a fluid biocompatable adhesive that reacts to the adhesive initiator. The material 120 may include any suitable initiator, as will be discussed in more detail herein.

It will be appreciated that the material 120 may include, for example, one or a plurality of initiators operably configured to activate, polymerize, or otherwise initiate the adhesive properties of an adhesive 146. Control of the molecular weight distribution of the applied adhesive 146 may be enhanced by selection of the concentration and functionality of the initiator or accelerator vis-a-vis the selected monomer. Suitable polymerization initiators and accelerators for cyanoacrylate compositions include, but are not limited to, base compositions, detergent compositions; surfactants, including nonionic surfactants such as polysorbate 20 (e.g. marketed under TWEEN 20 a trademark of ICI Americas), polysorbate 80 (e.g. marketed under TWEEN 80 a trademark of ICI Americas), and poloxamers; cationic surfactants such as tetrabutylammonium bromide; anionic surfactants, including quaternary ammonium halides such as benzalkonium chloride or its pure components, and benzethonium chloride; stannous octoate (tin (II)2-ethylhexanoate), and sodium tetradecyl sulfate; and amphoteric or zwitterionic surfactants such as dodecyldimethyl(3-sulfopropyl)ammonium hydroxide, inner salt; amines, imines, and amides, such as imidazole, tryptamine, urea, arginine and povidine; phosphines, phosphites and phosphonium salts, such as triphenylphosphine and triethyl phosphite; alcohols such as ethylene glycol; methyl gallate; inorganic bases and salts, such as sodium bisulfite, magnesium hydroxide, calcium sulfate and sodium silicate; sulfur compounds such as thiourea and polysulfides; polymeric cyclic ethers such as monensin, nonactin, crown ethers, calixarenes and polymeric epoxides; cyclic and acyclic carbonates, such as diethyl carbonate; phase transfer catalysts (e.g. marketed under ALIQUAT, a trademark of General Mills, Inc.); organometallics; manganese acetylacetonate; radical initiators and radicals, such as di-t-butyl peroxide and azobisisobutyronitrile; and bioactive compounds or agents. Other examples of adhesives 146 and adhesive initiators that may constitute the material 120 may be found in United States Publication 2004/0190975 to Goodman et al. which is herein incorporated by reference in its entirety.

Referring to FIG. 3, shown is a partial perspective view of tissue 90 having applied surgical staples 100. In the illustrated version, the surgical staples 100 are shown after the adhesive 146 has been applied thereto and after the adhesive has been activated after coming into contact with an initiator. The initiator, for example, may be associated with the material 120, shown in FIG. 1, where the surgical staples 100 may be doped or impregnated with the material 120, as discussed herein. The adhesive, as shown, may be polymerized or otherwise activated in regions substantially proximate or adjacent the surgical staples 100. For example, the adhesive 146 may be applied in a wash, as shown with reference to FIG. 2, where only portions of the adhesive 146 coming into contact with the initiator will solidify, become initiated, and/or demonstrate agglutinative properties. In a dilute application, or if an adhesive is provided having a low viscosity, only those regions substantially adjacent the surgical staples 100 may be initiated when the entire region of tissue 90 is bathed indiscriminately in adhesive. Adhesive 146 washed over the tissue 90 that does not come into contact with the material 120 containing an initiator may simply be washed off or may remain substantially adhesively inert. In this manner, only desirable regions of tissue 90 may have active adhesive associated therewith including, for example, the portions of the surgical staples 100 that are exposed through the tissue 90.

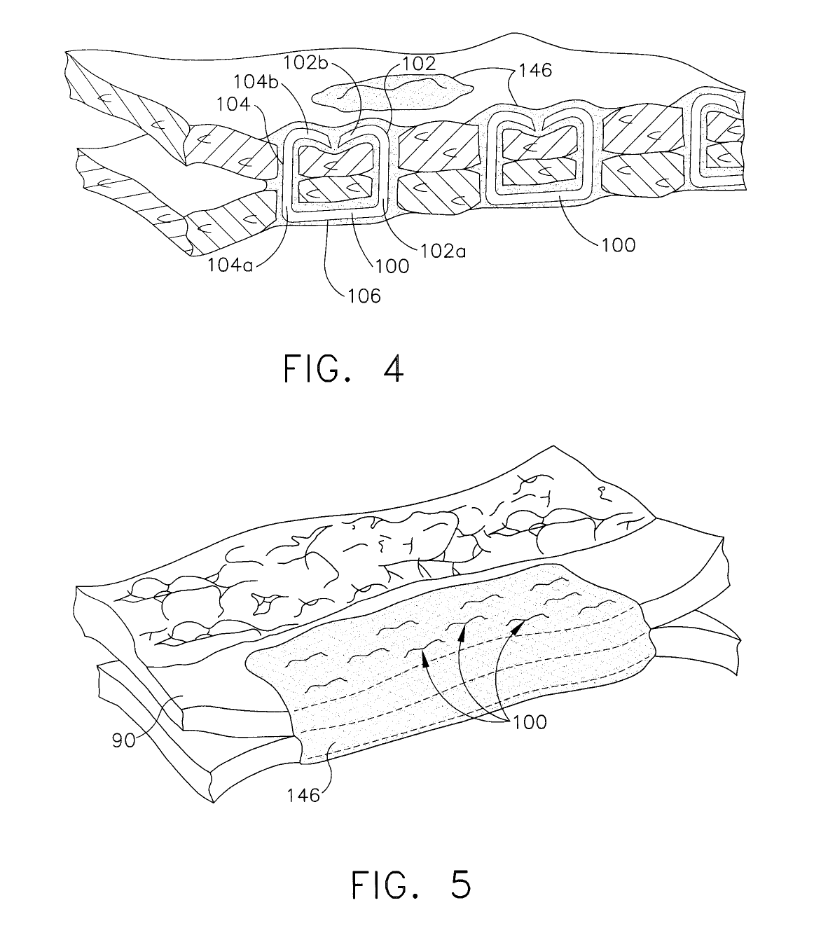

Referring to FIG. 4, shown is a cross-sectional view taken along line 4-4 of the portion of tissue 90 shown in FIG. 3. As illustrated, the adhesive 146 may be activated in only those regions substantially adjacent the surgical staples 100, where the material 120 containing an initiator has come into contact with the adhesive 146. In such a manner, the adhesive 146 may be activated in the most desirable areas, including the portions of the surgical staples 100 exposed through the tissue 90, to provide a seal or other advantageous tissue effect. At the same time, any superfluous adhesive 146 having a potentially negative affect on tissue, healing, and/or instrumentation may be reduced or eliminated by, for example, simply washing away the inactive adhesive 146 that has not come into contact with an initiator. Performing the method in accordance with FIGS. 3-4 may allow adhesive to be applied to tissue 90 in a substantially indiscriminate manner while still activating an adhesive in specific target areas. Such a method may combine the benefits of both adhesives and mechanical fasters while eliminating the disadvantages, such as instrument damage, associated with superfluous or uncontrolled adhesive.

FIG. 5 shows a partial perspective view of tissue 90 having applied surgical staples 100. In the illustrated version, the surgical staples 100 are shown after the adhesive 146 has been applied thereto and after the adhesive has been activated after coming into contact with an initiator. The initiator, for example, may be associated with the material 120, shown in FIG. 1, where the surgical staples 100 may be doped or impregnated with the material 120 as discussed herein. The adhesive, as shown, may be polymerized or otherwise activated such that a substantially contiguous adhesive layer is established about a plurality of surgical staples 100. For example, the adhesive 146 may be applied in a wash, as shown with reference to FIG. 2, where adhesive 146 coming into contact with the initiator will solidify or demonstrate agglutinative properties such that a layer of adhesive is created. The layer of adhesive 146 may be created, for example, by providing a more viscous adhesive than that described with reference to FIGS. 3-4 such that the adhesive is more likely to remain in the region of the initiator associated with the surgical staples 100. It will be appreciated that numerous methods for creating a substantially contiguous layer of adhesive in a desired region are contemplated where, for example, the region may be adhesively activated by providing a stronger or more potent initiator associated with the surgical staples, providing an adhesive 146 through which the initiator may readily creep, and/or providing an adhesive that is highly concentrated. As illustrated, a region adjacent the surgical staples 100 may be initiated when the entire region of tissue 90 is bathed in adhesive. Adhesive 146 washed over the tissue 90 not forming a portion of the layer of adhesive 146 may simply be washed off or may remain substantially adhesively inert. In this manner, patches or layers of adhesive in desirable areas may be created to, for example, join a plurality of surgical staples 100 with the application of substantially indiscriminate adhesive 146. As illustrated in FIG. 5, a layer of adhesive 146 may help seal a region of surgical staples 100 to facilitate healing, reduce the likelihood of infection, reduce or eliminate post-surgical bleeding, or otherwise provide a positive therapeutic effect.

It will be appreciated, with reference to all versions herein, that the initiator may be substituted with the adhesive in the various disclosed applications. For example, the surgical staples 100 may be provided with a material 120 having an adhesive impregnated, doped, or otherwise associated therewith. An initiator may then be administered from, for example, an initiator applicator to the region of adhesive such that the adhesive is activated in regions adjacent the surgical staples 100.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

What is claimed is:

1. A method for fastening tissue comprising the steps of:
providing a stapling instrument comprising a handle, a first and a second opposed tissue clamping member connected to the handle, the first and the second opposed tissue clamping members movable between an open position for receiving tissue and a closed position for stapling tissue therebetween, the first clamping member including a plurality of staples disposed therein in an array, the second clamping member comprising an anvil for forming the staples;
providing each of the plurality of staples with a portion of biocompatable material containing an adhesive initiator;
providing an adhesive applicator operably configured to deliver a portion of biocompatable material containing a fluid adhesive that is inactive, where the fluid adhesive is operably configured for activation by the adhesive initiator upon contact;
applying the plurality of staples to tissue;
providing the fluid adhesive in the region of the plurality of staples in an indiscriminate manner;
activating the fluid adhesive in regions adjacent the plurality of staples in which the fluid adhesive is associated with the adhesive initiator.

2. The method of claim 1, wherein the fluid adhesive and the adhesive initiator are bioabsorbable.

3. The method of claim 1, wherein the plurality of staples are bioabsorbable.

4. The method of claim 1, wherein the adhesive is selected from the group consisting of a polymerizable monomer, a polymerizable 1,1,1,1-disubstituted ethylene monomer, a cyanoacrylate formulation, and combinations thereof.

5. The method of claim 1, wherein the fluid adhesive is configured in a concentration such that isolated regions of activated adhesive are formed adjacent each of a plurality of the staples.

6. The method of claim 1, wherein the adhesive is configured in a concentration such that regions of activated adhesive are formed adjacent a plurality of the staples.

7. The method of claim 1, further comprising the step of washing away inactive fluid adhesive from the tissue.

8. The method of claim 1, wherein the fluid adhesive is configured for initiation at about a crown and at about a first leg and a second leg of each of the plurality of surgical staples.

9. The method of claim 1, wherein the step of providing the fluid adhesive further comprises applying the fluid adhesive to an external surface of tissue and to an internal surface of tissue.

10. A method for fastening tissue comprising the steps of:
providing a stapling instrument comprising a handle, first and a second opposed tissue clamping members connected to the handle, the first and a second opposed tissue clamping members movable between an open position for receiving tissue and a closed position for stapling tissue therebetween, the first clamping member including a plurality of staples disposed therein in an array, the second clamping member comprising an anvil for forming the staples;
providing a staple having a portion of biocompatable material containing an inactive adhesive;
providing an initiator applicator operably configured to deliver a portion of biocompatable material containing an adhesive initiator, wherein the adhesive initiator is operably configured to activate the adhesive;
applying the plurality of staples to tissue;
providing the adhesive initiator in the region of the plurality of staples in an indiscriminate manner;
activating the adhesive in regions adjacent the plurality of staples in which the adhesive is associated with the adhesive initiator.

11. The method of claim 10, wherein the adhesive and the adhesive initiator are bioabsorbable.

12. The method of claim 10, wherein the staples are bioabsorbable.

13. The method of claim 10, wherein the adhesive is selected from the group consisting of a polymerizable monomer, a polymerizable 1,1,1,1-disubstituted ethylene monomer, a cyanoacrylate formulation, and combinations thereof.

14. The method of claim 10, wherein the initiator is configured in a concentration such that isolated regions of activated adhesive are formed adjacent each of a plurality of the staples.

15. The method of claim 10, wherein the adhesive and the adhesive initiator are configured in a concentration such that regions of activated adhesive are formed adjacent at least two of the plurality of staples.

16. The method of claim 10, further comprising the step of washing away inactive fluid adhesive from the tissue.

17. The method of claim 10, wherein the adhesive is configured for initiation at about a crown and at about a first leg and a second leg of each of the plurality of surgical staples.

18. The method of claim 1, wherein the step of providing the adhesive initiator further comprises applying the adhesive initiator to an external surface of the tissue and to an internal surface of the tissue.
